(19) United States
(12) Patent Application Publication
(10) Pub. No.: US 2021/0342195 A1
(43) Pub. Date: Nov. 4, 2021

(54) MEDICAL IMAGE ANALYSIS DEVICE, METHOD, AND PROGRAM

(71) Applicant: FUJIFILM Corporation, Tokyo (JP)

(72) Inventor: Yuya KUDO, Tokyo (JP)

(73) Assignee: FUJIFILM Corporation, Tokyo (JP)

(21) Appl. No.: 17/378,761

(22) Filed: Jul. 18, 2021

Related U.S. Application Data
(63) Continuation of application No. PCT/JP2019/044388, filed on Nov. 12, 2019.

(30) Foreign Application Priority Data
Jan. 30, 2019 (JP) 2019-014455

(51) Int. Cl. G06F 9/50; G06T 7/00; G06T 1/60

(52) U.S. Cl. CPC G06F 9/5038; G06F 9/505; G06T 1/60; G06T 7/0012

(57) ABSTRACT

A part recognition unit recognizes a part included in a medical image. An analysis processing selection unit selects one or more tasks of analysis processing for analyzing the medical image from among a plurality of tasks of analysis processing based on the recognized part. An execution characteristic acquisition unit acquires, as execution characteristics, a usage amount of a resource and a processing time for each of a plurality of execution units included in the selected tasks of analysis processing. An allocation determination unit determines allocation of the plurality of execution units to a predetermined resource based on the execution characteristics, the execution unit common to the selected tasks of analysis processing, and a processing order of the plurality of execution units. An analysis execution unit executes analysis of the medical image by the selected tasks of analysis processing according to the determined allocation.

### CROSS REFERENCE TO RELATED APPLICATIONS

The present application is a Continuation of PCT International Application No. PCT/JP2019/044388 filed on Nov. 12, 2019, which claims priority under 35 U.S.C. § 119(a) to Japanese Patent Application No. 2019-014455 filed on Jan. 30, 2019. Each of the above applications is hereby expressly incorporated by reference, in its entirety, into the present application.

### BACKGROUND OF THE INVENTION

### Technical Field

The present disclosure relates to a medical image analysis device, a method, and a program for analyzing a medical image.

### Related Art

In the medical field, the performance of image analysis technology has improved. In particular, in recent years, the accuracy of analysis processing for recognizing lesions and specifying the lesions has improved by using artificial intelligence (AI) that uses a neural network trained via deep learning. A high-performance computer is required to execute such analysis processing.

On the other hand, various cloud services using a plurality of servers have become available in recent years. For example, services using the cloud have been provided without having expensive computers. The analysis processing using the AI described above is performed by using the cloud service, and thus, it is possible to leave the maintenance, operation, and management of hardware to a company that provides the cloud. As a result, users of the cloud services can reduce costs and human resources.

In the cloud service described above, it is easy to secure resources. Here, examples of the resources include a CPU, a memory, a hard disk, a network, and the like. However, the usage fee of the cloud service becomes higher as the amount of resources used becomes larger. That is, although a lot of resources can be secured in the cloud service, the usage fee becomes high in a case where a lot of resources are used.

Thus, a method for efficiently using the resources has been proposed. For example, JP2017-068393A has proposed a method for predicting a processing time and resource consumption in a case where analysis processing on first data is executed in a processing device, predicting processing performance related to analysis processing on second data based on the predicted processing time and resource consumption, and selecting a processing device for executing the analysis processing on the second data from among a plurality of processing devices based on the predicted processing performance in a case where analysis processing for training a model which performs prediction and classification of data is executed. JP2010-165259A has proposed a method for allocating a workload to processing units based on allocation conditions such as the number of processing units required, a degree of priority of each processing unit, and the maximum number of allocated processing units in a case where a workload such as artificial intelligence constituted by a plurality of tasks is allocated to the processing units and executed.

On the other hand, the analysis processing is often prepared for each part such as lungs, heart, liver, and brain and is further prepared for each detectable lesion. The amount of resources required for processing is different for each task of analysis processing. The methods described in JP2017-068393A and JP2010-165259A can allocate appropriate resources for a plurality of tasks of processing. However, even though the methods described in JP2017-068393A and JP2010-165259A are used, in a case where it is necessary to perform the analysis processing that requires a lot of resources, it is necessary to secure a lot of resources. In a case where a lot of resources are secured in this manner and analysis processing can be executed with a relatively small number of resources, there is a large waste of resources.

### SUMMARY OF THE INVENTION

The present disclosure has been made in view of the above circumstances, and an object of the present disclosure is to ensure that resources are used without waste.

A medical image analysis device according to an aspect of the present disclosure is a medical image analysis device that analyzes a medical image by using a predetermined resource by means of a plurality of tasks of analysis processing for analyzing the medical image, the plurality of tasks of analysis processing each including one or more execution units.

The medical image analysis device includes an image acquisition unit that acquires at least one medical image, a part recognition unit that recognizes a part included in the at least one medical image, an analysis processing selection unit that selects one or more tasks of analysis processing for analyzing the at least one medical image from among the plurality of tasks of analysis processing based on the recognized part, an execution characteristic acquisition unit that acquires, as execution characteristics, a usage amount of resource and a processing time for each of a plurality of execution units included in the selected tasks of analysis processing, an allocation determination unit that determines allocation of the plurality of execution units to the predetermined resource based on the execution characteristics, the execution unit common to the selected tasks of analysis processing, and a processing order of the plurality of execution units, and an analysis execution unit that executes analysis of the at least one medical image by the selected tasks of analysis processing according to the determined allocation.

The “resource” refers to physical computer resources, such as CPU, memory, storage, and network.

In the medical image analysis device according to the aspect of the present disclosure, the allocation determination unit may determine the allocation of the plurality of execution units to the predetermined resource for minimizing the processing time.

In the medical image analysis device according to the aspect of the present disclosure, in a case where processing by at least one execution unit is completed, the allocation determination unit may re-determine allocation of an unexecuted execution unit other than the execution unit by which the processing is completed to the predetermined resource based on the execution characteristics, the execution unit common to the selected tasks of analysis processing, and the processing order of the plurality of execution units for the unexecuted execution unit, and the analysis execution unit may execute processing by the unexecuted execution unit according to the re-determination.

The medical image analysis device according to the aspect of the present disclosure may further include a storage unit that stores the execution characteristics for each of the plurality of execution units, in which the execution characteristic acquisition unit acquires the execution characteristics from the storage unit.

The medical image analysis device according to the aspect of the present disclosure may further include an allocation information generation unit that generates allocation information representing the determined allocation.

In the medical image analysis device according to the aspect of the present disclosure, the allocation information generation unit may generate the allocation information by arranging the execution characteristics represented by rectangular regions which define a relationship between the processing time and the usage amount of the resource on a coordinate plane which defines the relationship between the processing time and the usage amount of the resource according to the determined allocation.

In the medical image analysis device according to the aspect of the present disclosure, the allocation information generation unit may further generate processing queue information in which unprocessed execution characteristics represented by rectangular regions which define a relationship between the processing time and the usage amount of the resource are arranged on a time axis on a coordinate plane which defines the relationship between the processing time and the usage amount of the resource according to the determined allocation.

The medical image analysis device according to the aspect of the present disclosure may further include an output unit that outputs the allocation information.

A medical image analysis method according to an aspect of the present disclosure is a medical image analysis method for analyzing a medical image by using a predetermined resource by means of a plurality of tasks of analysis processing for analyzing the medical image, the plurality of tasks of analysis processing each including one or more execution units.

The medical image analysis method includes acquiring at least one medical image, recognizing a part included in the at least one medical image, selecting one or more tasks of analysis processing for analyzing the at least one medical image from among the plurality of tasks of analysis processing based on the recognized part, acquiring, as execution characteristics, a usage amount of resource and a processing time for each of a plurality of execution units included in the selected tasks of analysis processing, determining allocation of the plurality of execution units to the predetermined resource based on the execution characteristics, the execution unit common to the selected tasks of analysis processing, and a processing order of the plurality of execution units, and executing analysis of the at least one medical image by the selected tasks of analysis processing according to the determined allocation.

A program causing a computer to execute the medical image analysis method according to the present disclosure may be provided.

Another medical image analysis device according to an aspect of the present disclosure is a medical image analysis device that analyzes a medical image by using a predetermined resource by means of a plurality of tasks of analysis processing for analyzing the medical image, the plurality of tasks of analysis processing each including one or more execution units.

The medical image analysis device includes a memory that stores instructions to be executed by a computer, and a processor that is configured to execute the stored instructions. The processor acquires at least one medical image, recognizes a part included in the at least one medical image, selects one or more tasks of analysis processing for analyzing the at least one medical image from among the plurality of tasks of analysis processing based on the recognized part, acquires, as execution characteristics, a usage amount of resource and a processing time for each of a plurality of execution units included in the selected tasks of analysis processing, determines allocation of the plurality of execution units to the predetermined resource based on the execution characteristics, the execution unit common to the selected tasks of analysis processing, and a processing order of the plurality of execution units, and executes analysis of the at least one medical image by the selected tasks of analysis processing according to the determined allocation.

According to the present disclosure, the resource can be used without waste.

### BRIEF DESCRIPTION OF THE DRAWINGS

FIG. 1 is a schematic configuration diagram of a medical image analysis system to which a medical image analysis device according to an embodiment of the present disclosure is applied.

FIG. 2 is a schematic configuration diagram of the medical information system including a terminal device.

FIG. 3 is a schematic configuration diagram of the medical image analysis device according to the embodiment of the present disclosure.

FIG. 4 is a schematic diagram showing a plurality of tasks of analysis processing.

FIG. 5 is a schematic diagram showing execution units of the analysis processing.

FIG. 6 is a schematic diagram showing generation of execution characteristics.

FIG. 7 is a schematic diagram showing selected tasks of analysis processing.

FIG. 8 is a diagram illustrating determination of a processing order of the execution units.

FIG. 9 is a diagram showing a processing queue.

FIG. 10 is a diagram showing an allocation result of the execution units.

FIG. 11 is a diagram showing a medical image after the analysis processing.

FIG. 12 is a flowchart showing processing performed in the present embodiment.

FIG. 13 is a diagram showing a processing queue and an allocation result in another embodiment.

FIG. 14 is a schematic diagram showing another example of the selected tasks of analysis processing.

FIG. 15 is a diagram illustrating another example of the determination of the processing order of the execution units.

FIG. 16 is a diagram showing another example of the allocation result of the execution units.

### DETAILED DESCRIPTION

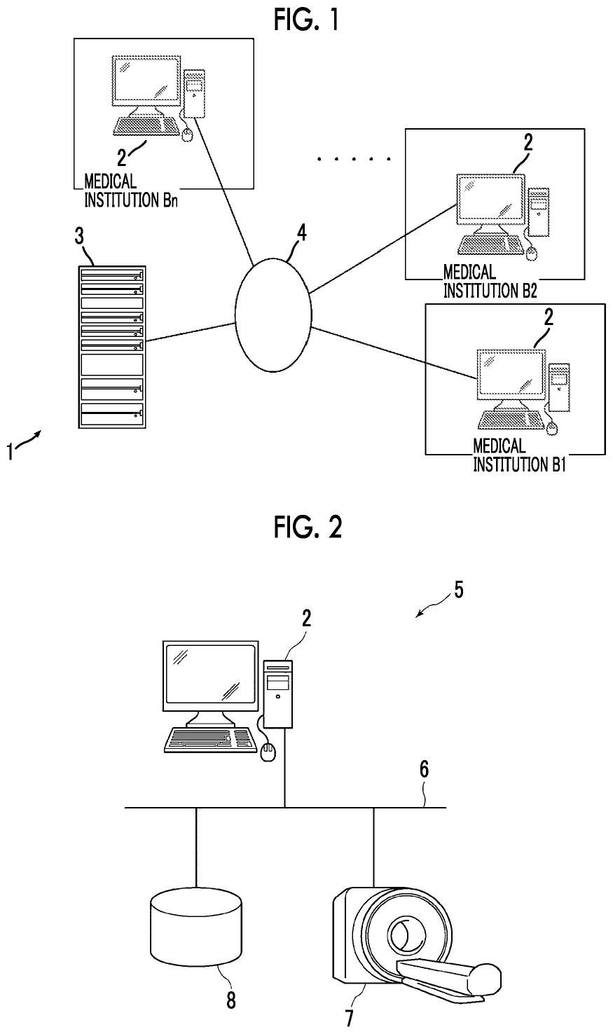

FIG. 1 is a schematic configuration of a medical image analysis system to which a medical image analysis device according to an embodiment of the present disclosure is applied. As shown in FIG. 1, a medical image analysis system 1 is configured such that a plurality of terminal devices 2 installed in a plurality of medical institutions Bk (k=1 to n) and a medical image analysis device 3 provided on a cloud side are connected via a network 4.

The terminal device 2 is a computer provided in each medical institution Bk, and is a well-known hardware such as a central processing unit (CPU), a memory, a storage, an input and output interface, a communication interface, an input device, a display device, and a data bus. A well-known operating system and the like are installed on the terminal device 2. The terminal device has a keyboard, a mouse, and the like as the input device and a display and the like as the display device. The medical image analysis device 3 according to the present embodiment will be described later.

The network 4 is a wide area network (WAN) that connects the terminal devices 2 provided in the plurality of medical institutions Bk and the medical image analysis device 3 over a wide area via a public network or a dedicated network.

FIG. 2 is a schematic configuration diagram of a medical information system including the terminal device 2. As shown in FIG. 2, the terminal device 2 is connected to a medical information system 5 of each medical institution Bk via a local area network (LAN) 6. The medical information system 5 includes a modality (imaging device) 7 and an image database 8, and is configured to transmit and receive image data to and from each other via the network 6.

The modality 7 includes a device that generates an inspection image representing an inspection target part of a subject by capturing the inspection target part, adds supplementary information defined by a DICOM standard to the inspection image, and outputs the inspection image. Specific examples include computed tomography (CT) devices, magnetic resonance imaging (MRI) devices, positron emission tomography (PET) devices, ultrasonic devices, computed radiography (CR) devices using a flat panel detector (FPD), and the like.

The image database 8 incorporates a software program that provides functions of a database management system to a general-purpose computer, and has a large-capacity storage. This storage may be a large-capacity hard disk device, or may be a disk device connected to a network attached storage (NAS) and a storage area network (SAN) connected to the network 6. The image data acquired in the modality 7 is transmitted to the image database 8 via the network 6 and is stored in accordance with a storage format and a communication standard conforming to the DICOM standard.

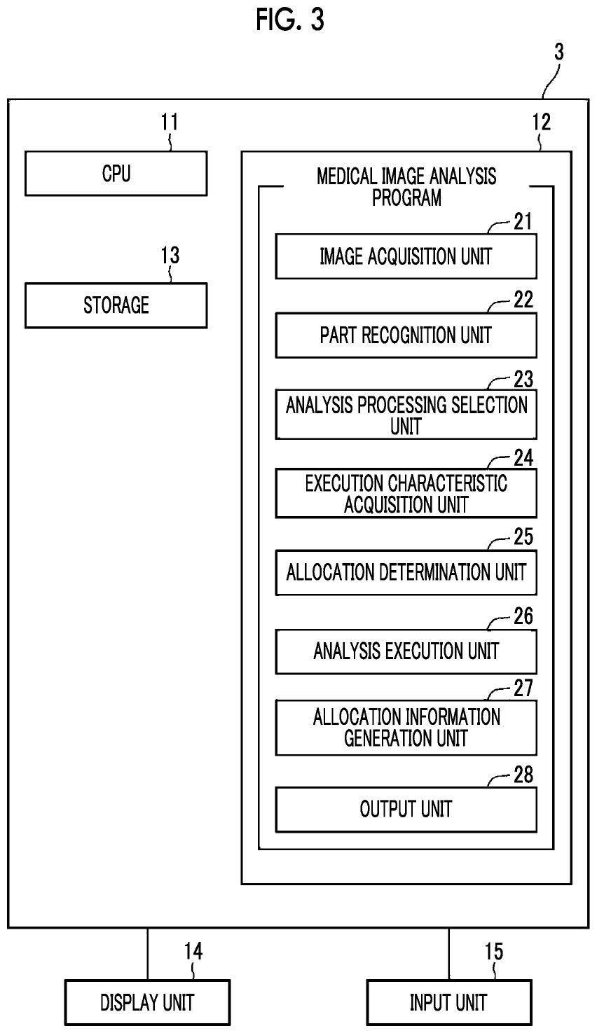

The medical image analysis device 3 is a computer for performing analysis processing on a medical image by means of a plurality of tasks of analysis processing by using a predetermined resource. FIG. 3 is a schematic configuration diagram of the medical image analysis device according to the embodiment of the present disclosure which is realized by installing a medical image analysis program on a computer. As shown in FIG. 3, the medical image analysis device 3 includes a CPU 11, a memory 12, and a storage 13. The medical image analysis device 3 includes an input and output interface (not shown), a communication interface, a data bus, and the like, has a well-known operating system and the like installed thereon, and has functions of a server. A graphics processing unit (GPU) may be provided in the medical image analysis device 3 as needed. The medical image analysis device 3 may be constituted by a plurality of computers.

A display unit 14 and an input unit 15 such as a mouse and a keyboard are connected to the medical image analysis device 3. In a case where the medical image analysis device 3 is constituted by the plurality of computers, each of the computers includes the CPU 11, the memory 12, and the storage 13, and the medical image analysis program is installed on each computer.

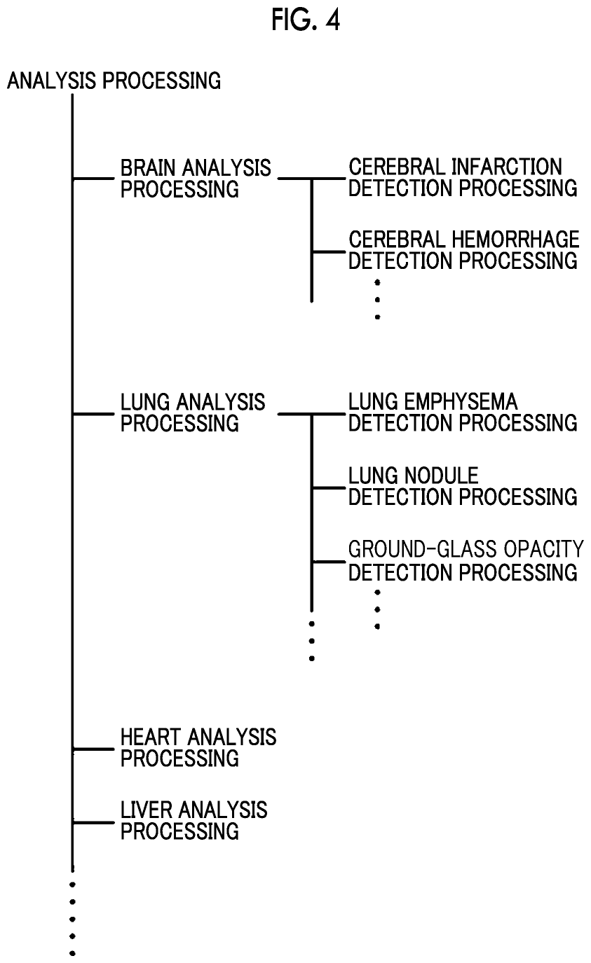

The plurality of tasks of analysis processing for analyzing the medical image are stored in the storage 13. The analysis processing is processing of analyzing the medical image and detecting a lesion or the like included in the medical image, and is constituted by, for example, AI. In the present embodiment, the plurality of kinds of analysis processing are stored in the storage 13 depending on parts of the human body. FIG. 4 is a schematic diagram showing the plurality of tasks of analysis processing. As shown in FIG. 4, in the present embodiment, analysis processing is prepared, as the analysis processing, for each of a plurality of parts, such as brain analysis processing, lung analysis processing, heart analysis processing, and liver analysis processing. The brain analysis processing includes analysis processing such as cerebral infarction detection processing and cerebral hemorrhage detection processing. The lung analysis processing includes analysis processing such as lung emphysema detection processing, lung nodule detection processing, and ground-glass opacity detection processing.

The storage 13 stores execution characteristics of each analysis processing. The execution characteristics will be described later. The storage 13 stores various kinds of information including medical images as targets of the analysis processing and information necessary for processing which are acquired from the terminal device 2 via the network 4.

The memory 12 stores the medical image analysis program. The medical image analysis program defines, as processing to be executed by the CPU 11, image acquisition processing of acquiring at least one medical image to be processed, part recognition processing of recognizing a part included in at least one medical image, analysis processing selection processing of selecting one or more tasks of analysis processing for analyzing at least one medical image from among the plurality of tasks of analysis processing based on the recognized part, execution characteristic acquisition processing of acquiring, as the execution characteristics, the usage amount of the resource and a processing time for each of a plurality of execution units included in the selected tasks of analysis processing, allocation determination processing of determining the allocation of the plurality of execution units to a predetermined resource based on the execution characteristics, an execution unit common to the selected tasks of analysis processing, and a processing order of the plurality of execution units, analysis execution processing of executing the analysis of at least one medical image by the selected tasks of analysis processing according to the determined allocation, allocation information generation processing of generating allocation information representing the determined allocation, and output processing of outputting the allocation information and an analysis result of the medical image to the terminal device 2 that has requested the processing.

The CPU 11 executes these tasks of processing according to the program, and thus, the computer functions as an image acquisition unit 21, a part recognition unit 22, an analysis processing selection unit 23, an execution characteristic acquisition unit 24, an allocation determination unit 25, an analysis execution unit 26, an allocation information generation unit 27, and an output unit 28. In a case where the medical image analysis device 3 is constituted by the plurality of computers, for example, one computer of the plurality of computers may execute all functions of the image acquisition unit 21, the part recognition unit 22, the analysis processing selection unit 23, the execution characteristic acquisition unit 24, the allocation determination unit 25, the analysis execution unit 26, the allocation information generation unit 27, and the output unit 28, and the other computers may execute only the function of the analysis execution unit 26.

The image acquisition unit 21 is constituted by, for example, a communication interface or the like, and acquires at least one medical image G0 to be analyzed, which is transmitted from the terminal device 2 via the network 4. Specifically, the medical image G0 is acquired by receiving the medical image G0 from the terminal device 2 of a doctor who wishes to analyze the medical image G0. In a case where the medical image G0 is already stored in the storage 13, the image acquisition unit 21 may acquire the medical image G0 from the storage 13. In the present embodiment, it is assumed that one medical image G0 is acquired.

The part recognition unit 22 recognizes a part of an inspected subject included in the medical image G0. For example, the part recognition unit 22 has a trained model trained to recognize the part included in the medical image G0. The trained model is generated by training a neural network with a large number of medical images of which parts are known as teaching data, and thus, in a case where the medical image G0 is input, the part included in the medical image G0 is output as a part recognition result. The part recognition unit 22 is not limited to a unit that recognizes the part via the trained model. For example, any method such as a method of recognizing the part included in the medical image G0 via template matching can be used as a part recognition method.

The analysis processing selection unit 23 selects one or more tasks of analysis processing for analyzing the medical image G0 from among the plurality of tasks of analysis processing stored in the storage 13 based on the part recognition result from the part recognition unit 22. For example, in a case where the medical image G0 is a medical image of the head, since the part recognition result is the brain, the analysis processing selection unit 23 selects one or more tasks of brain analysis processing. In a case where the medical image G0 is a medical image of an upper body of the human body, since the part recognition result is the heart and the lungs, the analysis processing selection unit 23 selects the heart analysis processing and the lung analysis processing.

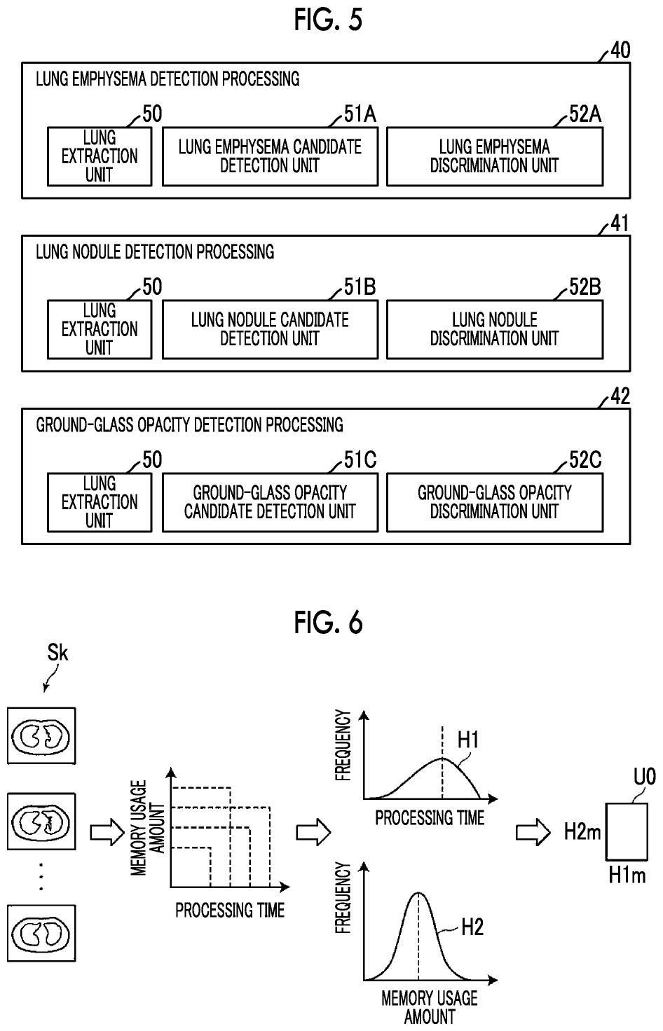

Here, the lung analysis processing includes a plurality of execution units such as extraction of the lungs from the medical image G0, detection of lesion candidates, and narrowing down of lesions, and can be divided into the execution units. FIG. 5 is a schematic diagram showing the execution unit of the analysis processing. In FIG. 5, lung emphysema detection processing 40, lung nodule detection processing 41, and ground-glass opacity detection processing 42 included in the lung analysis processing are shown schematically. The lung emphysema detection processing 40 shown in FIG. 5 is divided into three execution units of a lung extraction unit 50 for extracting the lungs from the medical image G0, a lung emphysema candidate detection unit 51A for detecting a lung emphysema candidate from the extracted lungs, and a lung emphysema discrimination unit 52A for discriminating whether or not the lung emphysema candidate is lung emphysema. The lung nodule detection processing 41 is divided into three execution units of a lung extraction unit 50, a lung nodule candidate detection unit 51B for detecting a lung nodule candidate from the extracted lungs, and a lung nodule discrimination unit 52B for discriminating whether or not the lung nodule candidate is a lung nodule. The ground-glass opacity detection processing 42 is divided into three execution units of a lung extraction unit 50, a ground-glass opacity candidate detection unit 51C for detecting a ground-glass opacity candidate from the extracted lungs, and a ground-glass opacity discrimination unit 52C for discriminating whether or not the ground-glass opacity candidate is ground-glass opacity. In FIG. 5, the execution units are arranged according to the processing order from the left side.

The execution characteristic acquisition unit 24 acquires, as the execution characteristics, the usage amount of the resource and the processing time for each of the plurality of execution units included in the selected tasks of analysis processing. Here, the resources are the CPU 11, the memory 12, and the storage 13 included in the medical image analysis device 3. An available resource usage amount is determined in advance according to the fee to be paid in each medical institution Bk. In the present embodiment, it is assumed that the capacity of the memory 12 available in the medical image analysis device 3 is determined in advance according to the usage fee.

In the present embodiment, the execution characteristics define a relationship between a memory usage amount and a processing time required for the execution unit of the analysis processing to perform the processing. The execution characteristics are generated in advance for each execution unit of the analysis processing and are stored in the storage 13. FIG. 6 is a schematic diagram showing the generation of the execution characteristics. Although the execution characteristic acquisition unit 24 generates the execution characteristics in the present embodiment, the execution characteristics may be generated by means provided separately from the execution characteristic acquisition unit 24. In the present embodiment, a plurality of sample images Sk for generating the execution characteristics are prepared as shown in FIG. 6. The execution characteristic acquisition unit 24 executes processing on each sample image Sk via the execution unit, and measures the processing time and the memory usage amount. The processing time and the memory usage amount vary depending on the sample image Sk.

Thus, the execution characteristic acquisition unit 24 generates histograms H1 and H2 representing frequencies for the processing time and a memory usage amount in a case where the execution unit performs processing on the plurality of sample images Sk. The execution characteristic acquisition unit 24 calculates an average value H1m of the histogram H1 for the processing time and an average value H2m of the histogram H2 for the memory usage amount, respectively, and generates, as the execution characteristics for the execution unit of the analysis processing, the average value H1m for the processing time and the average value H2m for the memory usage amount. The execution characteristics are shown schematically as a rectangle with a lateral axis as the average value H1m for the processing time and a vertical axis as the average value H2m for the memory usage amount. The generated execution characteristics are stored in the storage 13 in association with the execution unit of the analysis processing.

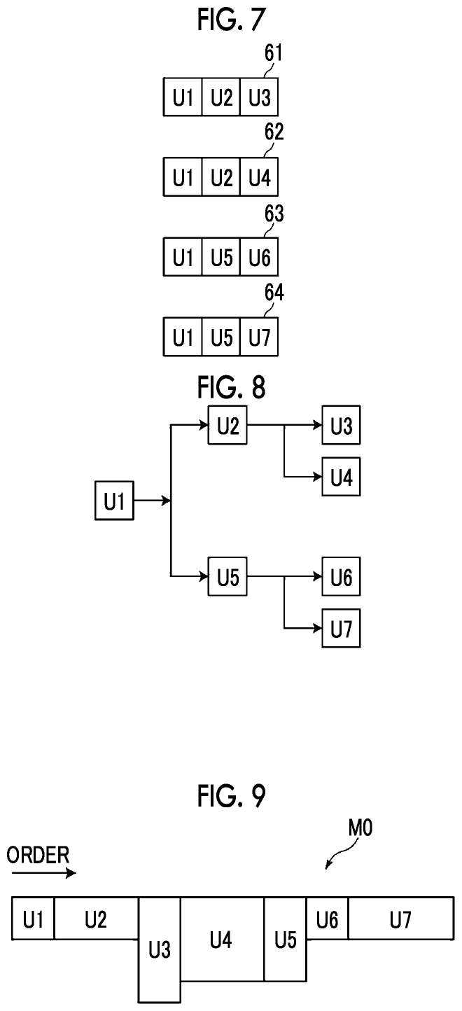

The allocation determination unit 25 determines the allocation of the plurality of execution units to the predetermined resource based on the acquired execution characteristics, the execution unit common to the selected tasks of analysis processing, and the processing order of the plurality of execution units. Hereinafter, the determination of the allocation will be described. In the present embodiment, it is assumed that four tasks of analysis processing 61 to 64 are selected as shown in FIG. 7. As shown in FIG. 7, the analysis processing 61 includes execution units U1, U2, and U3, the analysis processing 62 includes execution units U1, U2, and U4, the analysis processing 63 includes execution units U1, U5, and U6, and the analysis processing 64 includes execution units U1, U5, and U7. In FIG. 7, the execution units are arranged according to the processing order from the left side.

The allocation determination unit 25 specifies the execution unit common to the tasks of analysis processing 61 to 64. As shown in FIG. 7, the execution unit common to all the tasks of analysis processing 61 to 64 is the execution unit U1. The execution unit common to the tasks of analysis processing 61 and 62 is the execution unit U2. The execution unit common to the tasks of analysis processing 63 and 64 is the execution unit U5. Next, the allocation determination unit 25 specifies the processing order of the execution units. For example, in the execution unit U2 and the execution units U3 and U4, in a case where the execution unit U2 is not executed, the execution units U3 and U4 cannot be executed. In the execution unit U5 and the execution units U6 and U7, in a case where the execution unit U5 is not executed, the execution units U6 and U7 cannot be executed. On the other hand, the execution unit U2 and the execution unit U5 can perform the tasks of processing in parallel. The execution units U3, U4, U6, and U7 can perform the tasks of processing in parallel. Thus, the allocation determination unit 25 specifies the processing order of the execution units as shown in FIG. 8 in consideration of the processing order of the execution units in the selected tasks of analysis processing 61 to 64.

Subsequently, the allocation determination unit 25 determines the allocation of the plurality of execution units to the predetermined resource based on the execution unit common to the selected tasks of analysis processing and the processing order of the plurality of execution units. Thus, the allocation determination unit 25 generates a processing queue for the execution units. FIG. 9 is a diagram showing the processing queue. In a processing queue M0 shown in FIG. 9, it is assumed that each execution unit is represented by a rectangle having a size that reflects the execution characteristics, and a lateral axis defines the processing order. The allocation determination unit 25 allocates the execution units to the resource having a predetermined capacity while referring to the processing queue M0.

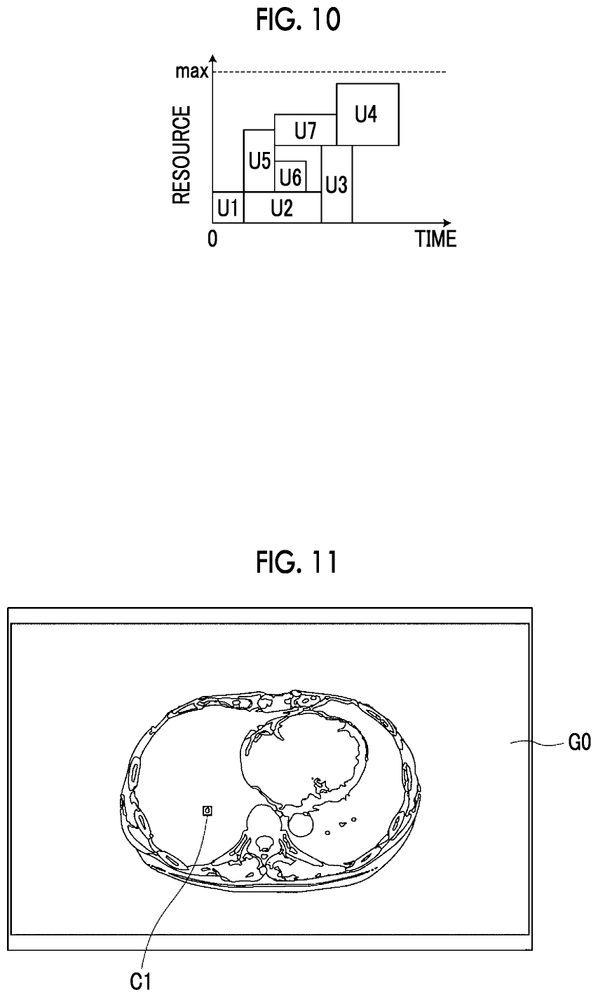

In the present embodiment, the allocation determination unit 25 determines the allocation of the plurality of execution units to the predetermined resource in order to minimize the processing time. Here, as shown in FIG. 9, in the processing queue M0, since the processing order of the execution units is U1, U2, U3, U4, . . . , the plurality of execution units are allocated such that the processing time is minimized while maximizing the use of the resource. FIG. 10 is a diagram showing an allocation result of the execution units. In FIG. 10, a vertical axis represents the resource, and in the present embodiment, represents the resource available to the medical institution Bk that has requested the analysis, that is, the capacity of the memory. The vertical axis also represents a maximum value max of the available capacity of the memory.

The analysis execution unit 26 executes the analysis of the medical image G0 by the selected tasks of analysis processing according to the allocation determined by the allocation determination unit 25. According to the allocation shown in FIG. 10, the analysis execution unit 26 first executes the execution unit U1 common to all the tasks of analysis processing 61 to 64, and executes the execution unit U2 common to the tasks of analysis processing 61 and 62 and the execution unit U5 common to the tasks of analysis processing 63 and 64 in parallel in a case where the processing of the execution unit U1 is ended. This is because the resource has a capacity capable of simultaneously executing the execution unit U2 and the execution unit U5. In the execution unit U2 and the execution unit U5, the processing of the execution unit U5 is ended first. Thus, in a case where the processing of the execution unit U5 is ended, the analysis execution unit 26 executes the execution unit U6 performed after the execution unit U5 in the analysis processing 63. Since the resource is free even though the execution units U2 and U6 are executed, the analysis execution unit 26 executes the execution unit U7 performed after the execution unit U5 in the analysis processing 64. In a case where the processing of the execution unit U2 is ended, the analysis execution unit 26 executes the execution unit U3 performed after the execution unit U2 in the analysis processing 61. In this state, the execution unit U7 is being executed, and there is no free resource in which the execution unit U4 is executable while the execution unit U3 and the execution unit U7 are being executed. Thus, the analysis execution unit 26 does not execute the execution unit U4 that is executed after the execution unit U2 in the analysis processing 62. In a case where the processing of the execution unit U7 is ended, since there is a free resource in which the execution unit U4 is executable, the analysis execution unit 26 executes the execution unit U4.

The allocation information generation unit 27 generates the allocation information representing the determined allocation. The allocation information is an image representing the allocation result shown in FIG. 10 determined by the allocation determination unit 25. The allocation information generation unit 27 may generate the processing queue as processing queue information. The processing queue information is an image representing the processing queue M0 shown in FIG. 9.

The output unit 28 is, for example, a communication interface or the like, and transmits the allocation information generated by the allocation information generation unit 27 to the terminal device 2 of the doctor who has requested the analysis of the medical image G0 via the network 4. In a case where the processing queue information is generated, the output unit 28 also transmits the processing queue information to the terminal device 2. Accordingly, a display unit of the terminal device 2 of the doctor displays the allocation information representing the allocation result shown in FIG. 10. In a case where the analysis of the medical image G0 by the analysis execution unit 26 is ended, the output unit 28 transmits the analysis result to the terminal device 2. The analysis result is information representing a position of an abnormal part in the medical image G0. Due to the use of this information, the terminal device 2 displays the medical image G0 in which the abnormal part is specified as shown in FIG. 11. As shown in FIG. 11, an abnormal part C1 is detected in the medical image G0.

Although the image acquisition unit 21 and the output unit 28 are provided separately in the present embodiment, the image acquisition unit 21 may have the function of the output unit 28, and the output unit 28 may have the function of the image acquisition unit 21.

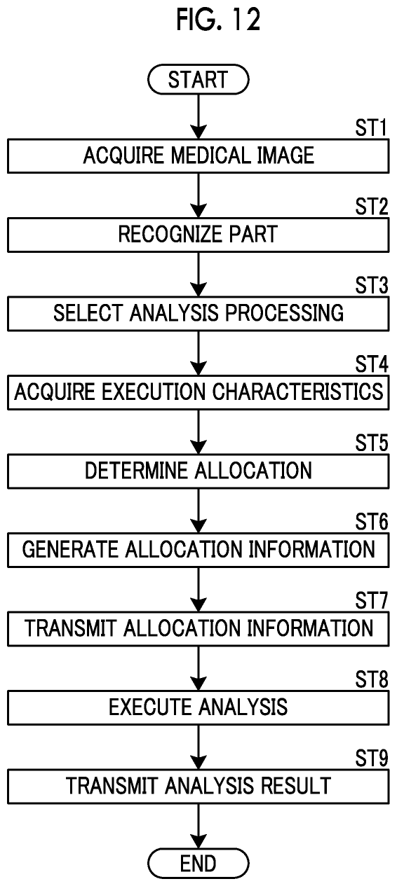

Next, processing performed in the present embodiment will be described. FIG. 12 is a flowchart showing the processing performed in the present embodiment. The image acquisition unit 21 acquires the medical image G0 to be analyzed, which is transmitted from the terminal device 2 of the medical institution Bk (step ST1). Subsequently, the part recognition unit 22 recognizes the part of the inspected subject included in the medical image G0 (step ST2). Subsequently, the analysis processing selection unit 23 selects one or more tasks of analysis processing for analyzing the medical image G0 from among the plurality of tasks of analysis processing stored in the storage 13 based on the part recognition result from the part recognition unit 22 (step ST3).

Subsequently, the execution characteristic acquisition unit 24 acquires, as execution characteristics, the usage amount of the resource and the processing time for each of the plurality of execution units included in the selected tasks of analysis processing from the storage 13 (step ST4). The allocation determination unit 25 determines the allocation of the plurality of execution units to the predetermined resource based on the acquired execution characteristics, the execution unit common to the selected tasks of analysis processing, and the processing order of the plurality of execution units (step ST5). The allocation information generation unit 27 generates the allocation information representing the determined allocation (step ST6), and the output unit 28 transmits the allocation information to the terminal device 2 (step ST7). On the other hand, the analysis execution unit 26 executes the analysis of the medical image G0 by the selected tasks of analysis processing according to the determined allocation (step ST8). The processing of step ST8 may be performed earlier than the tasks of processing of steps ST6 and ST7, and the tasks of processing of steps ST8 and ST6 and the processing of step ST7 may be performed in parallel. The output unit 28 transmits the analysis result to the terminal device 2 (step ST9), and ends the processing.

As described above, according to the present embodiment, the part included in the medical image G0 is recognized, and one or more tasks of analysis processing are selected from among the plurality of tasks of analysis processing based on the recognized part. The usage amount of the resource and the processing time for each of the plurality of execution units included in the selected tasks of analysis processing are acquired as the execution characteristics. The allocation of the plurality of execution units to the predetermined resource is determined based on the execution characteristics, the execution unit common to the selected tasks of analysis processing, and the processing order of the plurality of execution units, and the analysis of the medical image is executed by the selected tasks of analysis processing according to the determined allocation. Thus, according to the present embodiment, the execution units can be allocated to the resource such that there is less waste of resource. Accordingly, according to the present embodiment, the resource can be used without waste.

The allocation information representing the allocation result is generated and transmitted to the terminal device 2. Thus, the terminal device 2 can recognize how the execution unit is allocated to the resource and how the analysis processing is executed. At the medical institution Bk, it is possible to consider that increasing or decreasing the resource by checking the allocation information, and as a result, the capacity of the resource suitable for the analysis processing performed by the medical institution can be easily determined.

Although the analysis is executed according to the determined allocation in the above embodiment, in a case where the processing by at least one execution unit is completed, the allocation determination unit 25 may re-determine the allocation of the unexecuted execution unit to the predetermined resource based on the execution characteristics for an unexecuted execution unit other than the execution unit by which the processing is completed, the execution unit common to the selected tasks of analysis processing, and the processing order of the plurality of execution units. In this case, the analysis execution unit 26 executes the processing by the unexecuted execution unit according to the re-determination. Hereinafter, such allocation of the execution units will be described as another embodiment.

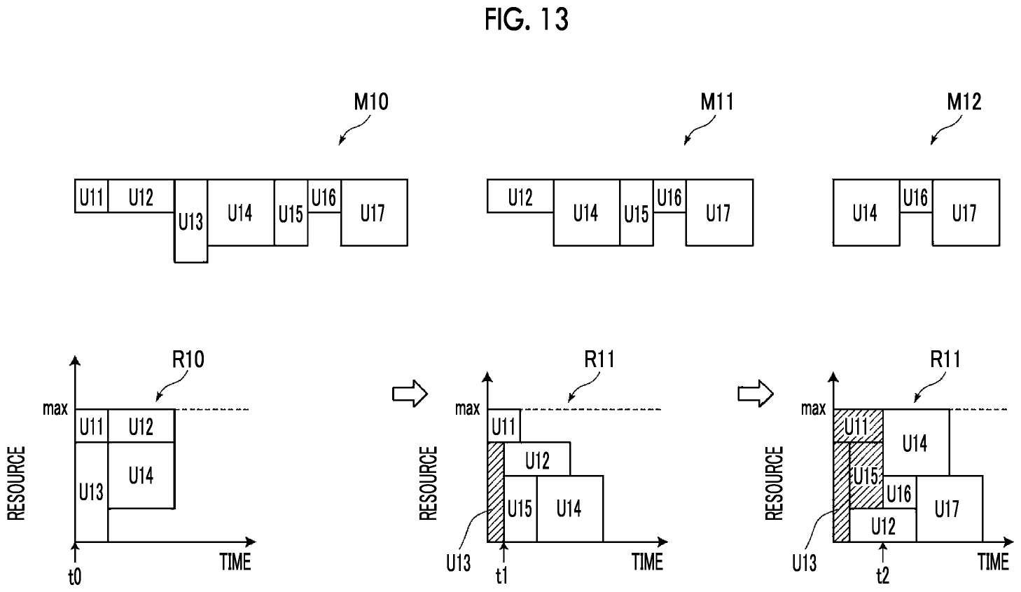

FIG. 13 is a diagram illustrating the allocation of the execution units in another embodiment. In FIG. 13, the processing queue and the allocation are shown in association with each other vertically. In another embodiment, it is assumed that the allocation of seven execution units U11 to U17 having execution characteristics is performed as shown in FIG. 13. First, the allocation determination unit 25 generates a processing queue M10 for the execution units U11 to U17, and allocates the execution units U11 to U17 to the resource according to the processing queue M10. An allocation result R10 is shown below the processing queue M10. The execution units U15 to U17 are not shown in the allocation result R10. An arrow indicates that a current processing time is an initial processing time t0 on a lateral axis of the allocation result R10. In the allocation result R10, the execution unit U11 and the execution unit U13 are executed in parallel, and the execution unit U12 and the execution unit U14 are executed in parallel after the execution unit U11 and the execution unit U13 are ended. The analysis execution unit 26 executes the analysis processing according to the allocation result R10. Accordingly, the analysis execution unit 26 first executes the execution unit U11 and the execution unit U13.

The execution unit Ulf and the execution unit U13 are executed, and thus, the allocation determination unit 25 generates a processing queue M11 by deleting the execution unit U11 and the execution unit U13 from the processing queue M10. On the other hand, it is assumed that the processing of the execution unit U13 is ended at time t1 earlier than planned in the analysis processing by the analysis execution unit 26. In FIG. 13, diagonal lines are given to the execution unit U13 by which the processing is ended. The allocation determination unit 25 re-determines the allocation of the execution unit while referring to the processing queue M11 based on the capacity of the resource after the execution unit U13 is ended. In an allocation result R11 obtained in this manner, the execution unit U12 and the execution unit U15 are performed earlier than the execution unit U14 after the execution unit U13 is ended. The execution units U16 and U17 are omitted in the allocation result R11. Accordingly, the analysis execution unit 26 continues to execute the execution unit U11, and executes the execution unit U12 and the execution unit U15 after the execution unit U13 is ended.

The execution unit U12 and the execution unit U15 are executed, and thus, the allocation determination unit 25 generates a processing queue M12 by deleting the execution unit U12 and the execution unit U15 from the processing queue M11. On the other hand, it is assumed that the execution unit U11 requires a longer time than planned, and the processing is ended at time t2 simultaneously with the execution unit U15 in the analysis processing by the analysis execution unit 26. In FIG. 13, diagonal lines are given to the execution units U11, U13, and U15 by which the processing is ended. The allocation determination unit 25 re-determines the allocation of the execution unit while referring to the processing queue M12 based on the capacity of the resource after the execution units U11 and U15 are ended. In an allocation result R12 obtained in this manner, after the execution units U11 and U15 are ended, execution regions of the execution unit U12 and the execution unit U14 are changed on the resource, and the execution unit U16 and the execution unit U17 are allocated. Accordingly, the analysis execution unit 26 continues to execute the execution unit U12, and executes the execution unit U14 and the execution unit U16 after the execution units U11 and U15 are ended. In a case where the execution unit U12 and the execution unit U16 are ended, the execution unit U17 is executed.

The allocation of the execution unit is re-determined in this manner, and thus, the execution unit can be efficiently allocated to the free resource even though the processing of the execution unit is ended earlier or later.

Although the analysis processing is performed on one medical image G0 in the above embodiment, the present disclosure can also be applied to a case where the analysis processing is performed on a plurality of medical images. For example, a case where three medical images G1 to G3 of a CT medical image G1 of the brain acquired by the CT device, an MRI medical image G2 of the brain acquired by the MRI device, and a CT medical image G3 of the chest acquired by the CT device are analyzed is considered.

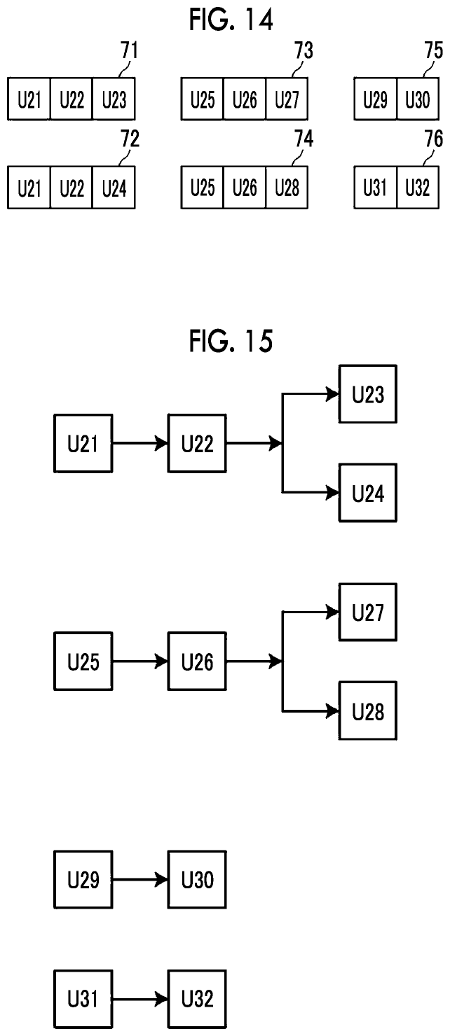

In this case, as shown in FIG. 14, it is assumed that cerebral infarction detection processing 71 for CT and cerebral hemorrhage detection processing 72 for CT are selected for the CT medical image G1 of the brain, cerebral infarction detection processing 73 for MRI and cerebral hemorrhage detection processing 74 for MRI are selected for the MRI medical image G2 of the brain, and lung analysis processing 75 for CT and heart analysis processing 76 for CT are selected for the CT medical image G3 of the chest.

As shown in FIG. 14, it is assumed that the cerebral infarction detection processing 71 for CT includes execution units U21, U22, and U23, and the cerebral hemorrhage detection processing 72 for CT includes execution units U21, U22, and U24. It is assumed that the cerebral infarction detection processing 73 for MRI includes execution units U25, U26, and U27, and the cerebral hemorrhage detection processing 74 for MRI includes execution units U25, U26, and U28. It is assumed that the lung analysis processing 75 includes execution units U29 and U30, and the heart analysis processing 76 includes execution units U31 and U32.

In such a case, it is necessary to perform six tasks of analysis processing, and the allocation determination unit 25 specifies the execution unit common to the tasks of analysis processing as in the above embodiment. As shown in FIG. 14, the execution units common to the cerebral infarction detection processing 71 for CT and the cerebral hemorrhage detection processing 72 for CT are the execution units U21 and U22. The execution units common to the cerebral infarction detection processing 73 for MRI and the cerebral hemorrhage detection processing 74 for MRI are the execution units U25 and U26.

The allocation determination unit 25 specifies the processing order of the execution units as shown in FIG. 15 in consideration of the processing order of the execution units. That is, as for the cerebral infarction detection processing 71 for CT and the cerebral hemorrhage detection processing 72 for CT, the allocation determination unit 25 executes the tasks of processing in the order of the execution unit U21 and the execution unit U22, and specifies the processing order of the execution units such that the execution unit U23 and the execution unit U24 are executed in parallel after the execution unit U22 is ended. As for the cerebral infarction detection processing 73 for Mill and the cerebral hemorrhage detection processing 74 for MRI, the allocation determination unit 25 executes the tasks of processing in the order of the execution unit U25 and the execution unit U26, and specifies the processing order of the execution units such that the execution unit U27 and the execution unit U28 are executed in parallel after the execution unit U26 is ended. As for the lung analysis processing 75, the allocation determination unit 25 specifies the processing order of the execution units such that the tasks of processing are executed in the order of the execution unit U29 and the execution unit U30. As for the heart analysis processing 76, the allocation determination unit 25 specifies the processing order of the execution units such that the tasks of processing are executed in the order of the execution unit U31 and the execution unit U32.

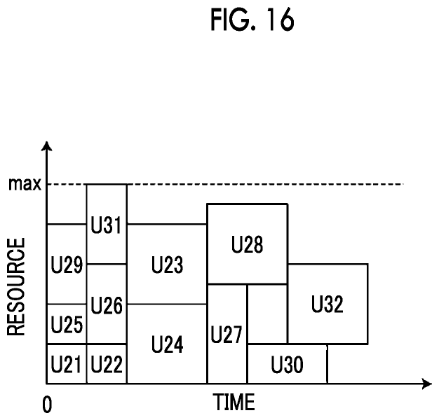

The allocation determination unit 25 determines the allocation of the plurality of execution units to the predetermined resource based on the execution unit common to the selected tasks of analysis processing and the processing order of the plurality of execution units. FIG. 16 is a diagram showing the allocation result of the execution units. The analysis execution unit 26 analyzes the medical images G1 to G3 by means of the selected tasks of analysis processing according to the allocation determined by the allocation determination unit 25.

Although the analysis processing is divided into minimum execution units in the above embodiment, the present disclosure is not limited thereto. The analysis processing may be divided such that the plurality of minimum execution units become one execution unit.

Although two or more tasks of analysis processing are selected from among the plurality of tasks of analysis processing in the above embodiment, only one task of analysis processing may be selected. In this case, the allocation of the execution units included in one task of analysis processing is determined, and the analysis processing is performed.

Although the medical image analysis device 3 is provided on the cloud side in the above embodiment, the analysis of the medical image G0 may be performed by using the terminal device 2 in the medical institution Bk. In this case, the medical image analysis device 3 may be incorporated in the terminal device 2. Accordingly, in the terminal device 2 of the medical institution Bk, the analysis of the medical image G0 can be performed by efficiently using the resource of the terminal device 2.

In each of the above-described embodiments, the following various processors can be used as a hardware structure of processing units that execute various kinds of processing, such as the image acquisition unit 21, the part recognition unit 22, the analysis processing selection unit 23, the execution characteristic acquisition unit 24, the allocation determination unit 25, the analysis execution unit 26, the allocation information generation unit 27, and the output unit 28. As described above, in addition to the CPU which is a general-purpose processor that functions as various processing units by executing software (programs), the various processors include a programmable logic device (PLD), which is a processor capable of changing a circuit configuration after manufacture, such as a field programmable gate array (FPGA), and a dedicated electrical circuit, which is a processor having a circuit configuration specifically designed in order to execute specific processing such as an application specific integrated circuit (ASIC).

One processing unit may be constituted by one of these various processors, or may be constituted by a combination of two or more processors of the same type or different types (for example, a combination of a plurality of FPGAs or a combination of a CPU and an FPGA). The plurality of processing units may be constituted by one processor.

As an example in which the plurality of processing units are constituted by one processor, firstly, one processor is constituted by a combination of one or more CPUs and software as represented by computers such as clients and servers, and this processor functions as the plurality of processing units. Secondly, a processor that realizes the functions of the entire system including the plurality of processing units via one integrated circuit (IC) chip is used as represented by a system on chip (SoC). As described above, the various processing units are constituted by using one or more of the various processors as the hardware structure.

More specifically, an electric circuitry in which circuit elements such as semiconductor elements are combined can be used as the hardware structure of these various processors.

What is claimed is:

1. A medical image analysis device that analyzes a medical image by using a predetermined resource by means of a plurality of tasks of analysis processing for analyzing the medical image, the plurality of tasks of analysis processing each including one or more execution units, the medical image analysis device comprising at least one processor, wherein the processor is configured to:
acquire at least one medical image;
recognize a part included in the at least one medical image;
select one or more tasks of analysis processing for analyzing the at least one medical image from among the plurality of tasks of analysis processing based on the recognized part;
acquire, as execution characteristics, a usage amount of resource and a processing time for each of a plurality of execution units included in the selected tasks of analysis processing;
determine allocation of the plurality of execution units to the predetermined resource based on the execution characteristics, the execution unit common to the selected tasks of analysis processing, and a processing order of the plurality of execution units; and
execute analysis of the at least one medical image by the selected tasks of analysis processing according to the determined allocation.

2. The medical image analysis device according to claim 1,
wherein the processor is configured to determine the allocation of the plurality of execution units to the predetermined resource for minimizing the processing time.

3. The medical image analysis device according to claim 2,
wherein, in a case where processing by at least one execution unit is completed, the processor is configured to re-determine allocation of an unexecuted execution unit other than the execution unit by which the processing is completed to the predetermined resource based on the execution characteristics, the execution unit common to the selected tasks of analysis processing, and the processing order of the plurality of execution units for the unexecuted execution unit, and
execute processing by the unexecuted execution unit according to the re-determination.

4. The medical image analysis device according to claim 1, further comprising:
a storage that stores the execution characteristics for each of the plurality of execution units,
wherein the processor is configured to acquire the execution characteristics from the storage.

5. The medical image analysis device according to claim 1,
wherein the processor is configured to generate allocation information representing the determined allocation.

6. The medical image analysis device according to claim 5,
wherein the processor is configured to generate the allocation information by arranging the execution characteristics represented by rectangular regions which define a relationship between the processing time and the usage amount of the resource on a coordinate plane which defines the relationship between the processing time and the usage amount of the resource according to the determined allocation.

7. The medical image analysis device according to claim 1,
wherein the processor is configured to generate processing queue information in which unprocessed execution characteristics represented by rectangular regions which define a relationship between the processing time and the usage amount of the resource are arranged on a time axis on a coordinate plane which defines the relationship between the processing time and the usage amount of the resource according to the determined allocation.

8. The medical image analysis device according to claim 5,
wherein the processor is configured to output the allocation information.

9. A medical image analysis method for analyzing a medical image by using a predetermined resource by means of a plurality of tasks of analysis processing for analyzing the medical image, the plurality of tasks of analysis processing each including one or more execution units, the medical image analysis method comprising:
acquiring at least one medical image;
recognizing a part included in the at least one medical image;
selecting one or more tasks of analysis processing for analyzing the at least one medical image from among the plurality of tasks of analysis processing based on the recognized part;
acquiring, as execution characteristics, a usage amount of resource and a processing time for each of a plurality of execution units included in the selected tasks of analysis processing;
determining allocation of the plurality of execution units to the predetermined resource based on the execution characteristics, the execution unit common to the selected tasks of analysis processing, and a processing order of the plurality of execution units; and
executing analysis of the at least one medical image by the selected tasks of analysis processing according to the determined allocation.

10. A non-transitory computer-readable storage medium that stores a medical image analysis program causing a computer to execute a medical image analysis method for analyzing a medical image by using a predetermined resource by means of a plurality of tasks of analysis processing for analyzing the medical image, the plurality of tasks of analysis processing each including one or more execution units, the program causing the computer to execute procedures of:
acquiring at least one medical image;
recognizing a part included in the at least one medical image;
selecting one or more tasks of analysis processing for analyzing the at least one medical image from among the plurality of tasks of analysis processing based on the recognized part;
acquiring, as execution characteristics, a usage amount of resource and a processing time for each of a plurality of execution units included in the selected tasks of analysis processing;
determining allocation of the plurality of execution units to the predetermined resource based on the execution characteristics, the execution unit common to the selected tasks of analysis processing, and a processing order of the plurality of execution units; and
executing analysis of the at least one medical image by the selected tasks of analysis processing according to the determined allocation.
